(19) United States
(12) Patent Application Publication
(10) Pub. No.: US 2012/0114582 A1
(43) Pub. Date: May 10, 2012

(54) ANTIPERSPIRANT COMPOSITIONS

(76) Inventors: Stephen Norman Batchelor, Wirral (GB); Jason Richard Williams, Wirral (GB)

(21) Appl. No.: 13/377,623

(22) PCT Filed: Jun. 15, 2010
(86) PCT No.: PCT/EP10/58363

§ 371 Date: Dec. 12, 2011

(30) Foreign Application Priority Data
Jun. 22, 2009 (GB) 0910657.6

(51) Int. Cl. A61K 8/49; A61Q 15/00; A61K 8/58

(52) U.S. Cl. 424/65

(57) ABSTRACT

Antiperspirant compositions containing a pigment often exhibit a dull colour if an inorganic pigment is employed, but if many bright organic pigments are used, the pigment is subject to photodegradation and accordingly fades. By incorporating selected types of polycyclic organic pigment, namely an organic pigment containing condensed aromatic or heterocyclic ring system that is free from an azo substituent and does not comprise, a di or th-arylcarbonium, or a xanthene based pigment, the antiperspirant composition comprises a bright pigment that is resistant to photodegradation. The composition is preferably anhydrous and advantageously a firm stick.

The present invention relates to antiperspirant compositions and their preparation, and more particularly to the incorporation therein of a pigment.

### BACKGROUND

Humans need to regulate their body temperature, and the principal way in which excess temperature is removed is by sweating. However, some societies consider it to be undesirable for others to see sweating, or subsequently detect body malodours arising from the transformation of organic chemicals carried out with sweat, or at least except when indulging in vigorous exercise. As a consequence, an industry has emerged offering products that suppress localised sweating, and especially in those areas of the body such as in the axilla (armpit) where the concentration of sweat glands (eccrine glands) is especially dense. The non-therapeutic products that reduce the rate of sweating comprise one or more antiperspirant active materials, often but not exclusively astringent salts disposed within a carrier and formulated as either a liquid or lotion, optionally together with a propellant, a gel, a cream, or a firm stick. The products can be anhydrous or comprise water, at the discretion of the manufacturer, depending on the properties of the product that it is desired to produce.

Although the effectiveness of an antiperspirant to control sweating is important to a user, it is not the only attribute which sways his or her decision as to which of the many competing products to buy. The aesthetics of the product are especially important as well. One of the most important aesthetic characteristic is the appearance of the product, not only in the dispenser, if visible, but particularly when dispensed. Users are very fussy about appearance, including its colour.

Although it is common for many antiperspirant products to be white, there are circumstances in which it is considered desirable for them to be coloured, either to enhance their distinctiveness to some customers, or include a colour to mask an otherwise undesirable colour, such as colour arising from one or more of the other ingredients in the composition, the colour arising from the ingredient itself or from an impurity. In order to achieve that, a coloured pigment can be incorporated. For the avoidance of doubt, the term colour herein does not include white. There is typically a choice from two classes of pigment, organic and inorganic. It is commonly considered preferable to incorporate an organic pigment, because such pigments tend to be brighter than inorganic pigments. However, many organic pigments suffer from photo-instability, by which is meant that the pigment degrades when exposed to sunlight, thereby losing its colour progressively, for example during storage or even when topically applied.

The photostability of an organic pigment depends on a wide variety of factors such as diffusion (rotational and translational), polarity and presence of catalysts. Consequently, its resistance to degradation varies significantly depending on its environment and thus on the other ingredients of the compositions it is intended to colour. For example, and without being bound by any particular theory, other ingredients can be catalytic in their own right or comprise catalytic impurities, such as various transition metal impurities. Antiperspirant compositions contain essentially an antiperspirant active, typically an aluminium and/or zirconium compound (eg a halohydrate) at a concentration that is high enough to impart localised perspiration-suppression when applied topically. Such materials are not only metal salts in their own right but commonly comprise various impurities such as other transition metals and halides. Consequently, the skilled person cannot predict whether a particular type of organic pigment would be photostable in an antiperspirant composition even if it were photostable when incorporated in some different composition.

Thus, although it would be logically desirable to incorporate a bright pigment into an antiperspirant composition when it is desired to colour it, it does not follow that such a pigment would remain photo-stable, even if in other types of composition it exhibited stability. For example, azo pigments are commonly employed in paints, products which are usually exposed to light and often to bright sunlight, on account of their bright colours. Such pigments are bright cost-effective and flexible pigments and for example the azo lake, Pigment Red 273 (CI 16035:1), has been used in antiperspirant stick products (Gillette Soft & Dri Clear Glide, floral Bouguet). However, current investigations have demonstrated a major drawback of azo pigments in antiperspirant sticks, namely that on exposure to sunlight they fade, sometimes rapidly. Thus, even though they might be predicted to be inherently suitable, in practice they are not.

Many patent applications have been published describing cosmetic compositions containing long lists of possible or optional classes of ingredients. Some of them mention as one possible class of ingredient within that list an antiperspirant or deodorant without disclosing any worked examples of compositions containing it. Within the set of publications relating to cosmetic compositions contemplating such inclusion of antiperspirant or deodorant, some merely make passing reference to the class alone and others disclose a number of possible active materials within the class. Many patent specification describing cosmetic compositions include as a class of ingredients within a long list of possible classes of ingredients dyes or pigments. Some of them simply disclose the possible incorporation of the class by name alone without any further disclosure and others describe a large or small number of pigments and/or dyes, typically including both organic and inorganic pigments if detailed disclosure is given.

Thus by way of example several patent specifications including US2007/0253922, US2004/0170670, US2005/0142085, and US2009/006849 disclose a pigment or dye as a possible ingredient within the list of possible ingredients and identify by name as an example of polycyclic organic pigments anthraquinone pigments or dyes. There is no express disclosure of any composition in such publications of a composition containing both an antiperspirant and an anthraquinone pigment. In fact, were such a composition containing such two selected classes of material be made, they would be comparatively undesirable because the resultant colour intensity is undesirably low unless a substantial concentration of the pigment were employed. It remains inherently desirable to avoid employing materials of comparatively poor colour intensity, not only to avoid the increased cost arising from a high concentration, but also because it deprives the formulator of formulation space. Such patent specifications do not disclose the problem of selecting a pigment that remains stable in the presence of an effective concentration of an antiperspirant active.

Other patent specifications including WO2007/077541, WO2009/034537, WO2006/1206446, US2009/0017147, WO2007/129270, WO2008/015639 and WO2007/029187 disclose the same list of 40 classes of active materials. Two of the classes within that list comprise antiperspirants and particulate material (13), without any express link between them. Within the class of particulate material, there are many different sub-classes, including coloured and uncoloured pigments, organic powders, composite powders, optical brightener particles and combinations thereof. Within class 13 there is also reference to filler powders. Manifestly, the sub-classes of particulate materials are suitable for many different purposes. There is no express teaching to select the sub-sub-class of pigments from the sub-class of particulate materials in the presence of an antiperspirant. There are many different pigment alternatives disclosed within its sub-sub-class, and there is no express teaching to select any particular pigment material type from within the pigments, let alone any particular pigment itself for employment together with an antiperspirant active.

In a co-pending application, PCT Application No PCT/EP 2008/067644, priority date 20 Dec. 2007, it is disclosed that the rate of degradation of an ingredient that is susceptible to photo-degradation can be reduced in antiperspirant compositions by incorporating a pigment that has a colour within 150 nm of the peak coefficient of extinction of the susceptible ingredient, and in other words matches the pigment to the susceptible ingredient. The specification discloses compositions in which the pigment is present in a weight ratio to the susceptible ingredient of at least 0.5:1. The disclosure therein does not recognise or identify which pigments are or are not particularly resistant to photo-destruction in such compositions. Accordingly, the specification does not teach the provision of antiperspirant compositions that are pigmented and comparatively colour stable.

### OBJECTS OF THE PRESENT INVENTION

It is an object of the present invention to devise pigmented antiperspirant compositions in which one or more of the foregoing disadvantages are ameliorated.

It is a further object of at least some of the embodiments of the present invention to devise antiperspirant compositions containing an organic coloured pigment that is resistant to photo-degradation.

### SUMMARY OF THE PRESENT INVENTION

According to one aspect of the present invention there is provided a non-therapeutic antiperspirant composition comprising
an antiperspirant active at a concentration of at least 5% by weight,
a carrier liquid for the antiperspirant active and
a polycyclic organic pigment selected from Cu phthalocyanine, metal free phthalocyanine, quinacridone, perylene, diketopyrrolo-pyrrole, thioindigo, anthrapyrimidine, flavanthrone, pyranthrone, dioxazine and quinophthalone pigments dispersed through the carrier liquid,

which composition is free from an ingredient that is susceptible to photo-degradation by visible and/or UV light having a peak coefficient of extinction that is within 150 nm of the colour of the pigment or contains less than 1 part by weight of said susceptible ingredient per 2 parts by weight of said pigment.

Herein by the term “pigment” is meant coloured particles visible to the human eye having a water solubility at 25° C. of <0.1% w/w. The particles preferably have a diameter of at least 0.02 μm, and commonly up to 10 μm.

Particle diameters are suitably measured by electron microscopy or ultrasedimentation, most preferably electron microscopy. The diameters refer to the primary particles.

Herein, the term “polycyclic organic pigment” means uncharged organic pigments which contain greater than 3 condensed aromatic or heterocyclic ring systems.

By employment of the selected sorts of pigments within the selected type of organic pigment, namely the polycyclic pigment, the pigment not only imparts colour to the composition, but it also is resistant to photodegradation in the presence of the antiperspirant active, thereby enabling the composition to retain colour for longer and at a greater intensity than if less-photoresistant organic pigments were to be employed. Advantageously, the selected organic pigments are capable of imparting bright colour to antiperspirant compositions.

### DETAILED DESCRIPTION OF THE PRESENT INVENTION AND PREFERRED EMBODIMENTS

The instant invention employs selected organic pigments that display a resistance to photodegradation in the presence of an antiperspirant active that is greater than that exhibited by azo pigments and azo lake, Pigment Red 273 (CI 16035:1) in particular.

Polycyclic organic pigments herein contain 4 or more, and preferably 5 or more condensed aromatic or heterocyclic ring systems.

In the context of the current invention polycyclic organic pigments have chemical structures which are uncharged. For example di and tri-arylcarbonium, and xanthene based pigment contain positive charges within their structures and are hence excluded.

Selected pigments for employment herein do not contain an azo chromophore (—N═N—).

Selected polycyclic organic pigments herein are derived from Cu phthalocyanine, metal free phthalocyanine, quinacridone, perylene, perinone, anthrapyrimidine, flavanthrone, pyranthone, isoviolanthrone, dioxazine and quinophthalone chromophores. A mixture of selected pigments herein can be employed, chosen either from the same sort of pigment, eg all phthalocyanine pigments, or from a mixture of different sorts. Consequently, the formulator can achieve a range of coloured antiperspirant products differing from the colour of individual pigments.

Examples of polycyclic organic pigments are Pigment Red 123 (perylene), Pigment Black 31 (perylene), Pigment Black 32 (perylene) Pigment Orange 43 (perinone), Pigment Orange 51 (pyranthrone), Pigment Orange 77 (flavanthrone), Pigment Violet 31 (isoviolanthrone), Pigment Blue 64 (indanthrone), Pigment Yellow 108 (anthrapyrimidine), Pigment Blue 60 (indanthrone), Pigment Yellow 24 (flavanthrone), Pigment Orange 40 (pyranthrone), Pigment Orange 51 (pyranthrone), Pigment Red 168 (anthranone), Pigment Violet 31 (isoviolanthrone), Pigment Violet 23 (dioxazine) and Pigment Yellow 138 (quinophthalone).

Particularly preferred polycyclic organic pigments are Cu phthalocyanine pigments, metal free phthalocyanine, pigments, quinacridone pigments and triphenodioxazine pigments.

Phthalocyanin pigments contain the phthalocyanine chromophore and may be metallated or unmetallated in accordance with the limitations disclosed hereinbefore. Examples include Pigment Blue 15, Pigment Blue 15:1, Pigment Blue 15:2, Pigment Blue 15:3, Pigment Blue 15:4, Pigment Blue 15:6, Pigment Blue 16, Pigment Blue 79, Pigment Green 7 and Pigment Green 36.

Quinacridone pigments contain the dioxotetrahydroquinolinoacridine chromophore. Preferably they are a five ring polycyclic system that maybe arranged in a linear or angular way. Examples include Pigment Violet 19 (beta and gamma modification), Pigment Red 122, Pigment Red 192, Pigment Red 202, Pigment Red 207 and Pigment Red 209.

Triphenodioxazine pigments contain the triphenodioxazine chromophore examples include Pigment Violet 23 and Pigment Violet 37.

Particularly preferred pigments are selected from Pigment Violet 23, Pigment Blue 60, Pigment Blue 64, Pigment Orange 43, Pigment blue 66, Pigment Blue 63, Pigment Violet 36, Pigment Violet 19, Pigment Red 122, Pigment Blue 16, Pigment Blue 15, Pigment Blue 15:1, Pigment Blue 15:2, Pigment Blue 15:3, Pigment Blue 15:4, Pigment Blue 15:6 and Pigment Green 7.

Other polycyclic pigments in accordance with the present invention can be found in “Industrial Organic Pigments, W. Herbst & K. Hunger (Wiley-VCH 2004 ISBN:3-527-30576-9). Some or other suitable pigments are listed in the Color Index International (C.I.) of the Society of Dyers and Colourists and American Association of Textile Chemists and Colorists.

The pigment may be applied uniformly through the base (by which herein is meant the ingredients of the composition other than the pigment and any propellant) to form a composition of a single colour, or non-uniformly in solidified compositions to give multi-coloured and/or patterned compositions.

Suitably, the antiperspirant compositions herein are anhydrous, by which is meant that the composition do not comprise an aqueous phase.

The weight proportion of the polycyclic pigment can be selected by the formulator to achieve the desired intensity of colour in the formulation. Commonly weight of pigment is selected in the range of from 0.001 to 1% of the total composition (excluding the weight of any propellant), especially at least 0.002% and particularly at least 0.004%. In many embodiments, a suitable intensity can be achieved at a pigment concentration of up to 0.2% and preferred compositions at up 0.05% by weight of propellant-free-composition.

The pigment can conveniently be pre-dispersed in a carrier liquid such as volatile silicone oil, for example at a concentration in the pre-mix of from 0.5 to 5% by weight, in order to assist dispersion within the eventual final composition.

Antiperspirant actives.

The composition preferably contains an antiperspirant active. Antiperspirant actives are preferably incorporated in an amount of from 5-50%, particularly from 5 to 30% and especially from 10% to 26% of the weight of the composition. It is often considered that the main benefit from incorporating of up to 5% of an antiperspirant active in a stick composition is manifest in reducing body odour, and that as the proportion of antiperspirant active increases, so the efficacy of that composition at controlling perspiration increases.

Antiperspirant actives for use herein are often and preferably selected from astringent active salts, including in particular aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates.

Aluminium halohydrates are usually defined by the general formula Al₂(OH)ₓQ_{y}.wH₂O in which Q represents chlorine, bromine or iodine, x is variable from 2 to 5 and x+y=6 while wH₂O represents a variable amount of hydration. Especially effective aluminium halohydrate salts, known as activated aluminium chlorohydrates, are described in EP-A-6739 (Unilever NV et al), the contents of which specification is incorporated herein by reference. Such activated aluminium chlorohydrates are made by a method in which the weight concentration of aluminium compounds in the solution is controlled within specified limits and simultaneously the temperature of that solution is controlled within a specified elevated temperature range whilst polymeric aluminium species are formed, and drying conditions are strictly controlled as described in the said EP-A-6739. Some activated salts do not retain their enhanced activity in the presence of water but are useful in substantially anhydrous formulations, i.e. formulations that do not contain a distinct aqueous phase.

Zirconium actives can usually be represented by the empirical general formula: ZrO(OH)_{2n-nz}B_{z}.wH₂O in which z is a variable in the range of from 0.9 to 2.0 so that the value 2n-nz is zero or positive, n is the valency of B, and B is selected from the group consisting of chloride, other halide, sulphamate, sulphate and mixtures thereof. Possible hydration to a variable extent is represented by wH₂O. Preferable is that B represents chloride and the variable z lies in the range from 1.5 to 1.87. In practice, such zirconium salts are usually not employed by themselves, but as a component of a combined aluminium and zirconium-based antiperspirant.

The above aluminium and zirconium salts may have co-ordinated and/or bound water in various quantities and/or may be present as polymeric species, mixtures or complexes. In particular, zirconium hydroxy salts often represent a range of salts having various amounts of the hydroxy group. Zirconium aluminium chlorohydrate may be particularly preferred.

Antiperspirant complexes based on the above-mentioned astringent aluminium and/or zirconium salts can be employed. The complex often employs a compound with a carboxylate group, and advantageously this is an amino acid. Examples of suitable amino acids include dl-tryptophan, dl-β-phenylalanine, dl-valine, dl-methionine and β-alanine, and preferably glycine which has the formula CH₂(NH₂)COOH.

It is highly desirable to employ complexes of a combination of aluminium halohydrates and zirconium chlorohydrates together with amino acids such as glycine, which are disclosed in U.S. Pat. No. 3,792,068 (Luedders et al). Certain of those Al/Zr complexes are commonly called ZAG in the literature. ZAG actives generally contain aluminium, zirconium and chloride with an Al/Zr ratio in a range from 2 to 10, especially 2 to 6, an Al/Cl ratio from 2.1 to 0.9 and a variable amount of glycine. Actives of this preferred type are available from Westwood, from Summit and from Reheis, though with differing particle size distributions.

Many aluminium and/or zirconium-containing astringent antiperspirant salts employed herein have metal:chloride mole ratio in the range of 1.3:1 to 1.5:1. Others having a lower metal:chloride mole ratio, such as from 1:1 to 1.25:1 tend to generate lower pHs when applied to skin and thus tend to be more irritating.

The proportion of solid antiperspirant salt in a suspension composition normally includes the weight of any water of hydration and any complexing agent that may also be present in the solid active.

Many particulate antiperspirants employed in the instant invention have a refractive index (RI) of at least 1.49 and not higher than 1.57. Actives which are free from zirconium tend to have an RI of from 1.49 to 1.54, depending on their formula and at least partly on their residual water content. Likewise, actives which contain zirconium tend to have an RI of from 1.52 to 1.57.

The selection of the antiperspirant active material desirably takes into account the type of applicator from which it is dispensed. Thus, in many particularly preferred embodiments in which the composition is dispensed from a contact applicator, for example using a stick, cream (soft solid) or roll-on dispenser, the antiperspirant active comprises an aluminium-zirconium active, such as AZAG. However, in other highly preferred embodiments in which the composition is dispensed as a spray, such as using an aerosol dispenser, the antiperspirant active is highly desirably an aluminium chlorohydrate (ACH) or an activated aluminium chlorohydrate (AACH).

The antiperspirant active employed herein comprises small particles, their average particle size and distribution commonly being selected in accordance with the nature of the applicator from which the composition is dispensed.

For incorporation of compositions according to the present invention, desirably at least 90%, preferably at least 95% and especially at least 99% by weight of the particles having a diameter in the range of from 0.1 μm up to 100 μm. For incorporation in contact applicators, such as stick, soft solid or roll-on dispensers, the antiperspirant particles usually have an average particle diameter of at least 1 μm and especially below 20 μm. In some highly desirable contact compositions, the particles by weight have an average particle size of at least 2 μm and particularly below 10 μm, such as in the range of from 3 to 8 μm.

For incorporation in non-contact applicators and especially in aerosols in which the composition is expelled from the dispenser by a propellant gas, possibly augmented by a mechanical or electromechanical propulsive means, it is especially desirable for less than 5% by weight, particularly less than 1% by weight and advantageously none of the particles to have a diameter of below 10 μm. Preferably for inclusion in aerosol compositions, the particles have a diameter of below 75 μm. In many preferred aerosol compositions, the antiperspirant has an average (D₅₀) particle diameter in the range of from 15 to 25 μm. The particle size of the antiperspirant active or mixture of actives can be measured using a Malvern Mastersizer, similarly to measurement of the perfume microcapsules size, as mentioned hereinbefore.

One method of seeking to minimise visible whiteness employs antiperspirant active material that is free or substantially free from hollow particles. In this context, substantially free indicates a content of less than 10% by weight hollow spheres, and preferably less than 5% by weight. Some drying techniques, e.g. spray drying, can produce materials which contain greater than such a proportion of hollow spheres, the proportion can be reduced by milling the particulate material, such as by ball or swing milling.

The proportion of antiperspirant active incorporated into the base composition is at the discretion of the formulator, and usually selected within the range of 5 to 60% by weight of the base composition, and often at least 10% by weight. In compositions free from a propellant, the weight proportion in the base composition is preferably not greater than 27% by weight and in many instances is selected in the range of from 10 to 25% by weight. In base compositions intended to be augmented by a propellant, the weight proportion of antiperspirant active (especially an aluminium chlorohydrate) is often selected in the range of from 5 to 50%, such as from 10 to 40%.

Commonly, the antiperspirant active is present in a weight ratio to the selected pigment of at least 60:1, such as up to 1000:1. In many suitable embodiments, the weight ratio is at least 100:1 and in many instances is up to 600:1. An especially practical range is from 15:1 to 450:1.

Carrier Liquid

The carrier liquid for the antiperspirant active can comprise one oil or a mixture oils, one or more of which can be classed as volatile and one or more of which can be classed as non-volatile. In accordance with customary usage in the field of antiperspirant compositions, an oil is considered to be volatile if it has a measurable vapour pressure at 25° C. of at least 1 Pa, and typically in a range of from 1 or 10 Pa to 2 kPa. The term “oil” herein means a water-immiscible material that has a melting point of or below 20° C. and a boiling point of greater than 100° C.

Volatile oils suitable for employment herein as carrier liquid (sometimes referred to as carrier fluid) include in particular volatile silicone oils. Such oils are commonly cyclomethicones (cyclodimethylsiloxanes) usually containing from 4 to 7 methicone units, which are conveniently designated as D4, D5, D6 or D7 respectively. Particularly desirably, the volatile oil comprises D5 and/or D6. Other suitable volatile silicone oils comprise short chain linear dimethicones, such as containing 3 or 4 to 7 silicon atoms, conveniently designated L3, L4, L5, L6 or L7 respectively. At the discretion of the formulator, the volatile silicone can comprise a mixture of cyclo and linear compounds.

Other volatile oils that can be contemplated instead of or in addition to volatile oils include volatile hydrocarbon oils, commonly branched hydrocarbons containing from 12 to 20 carbons. Preferably such oils comprise less than 5% by weight of the blend constituting the carrier liquid.

Non-volatile oils are commonly selected from silicone oils, ester oils, ether oils, hydrocarbon oils and alcohol oils that satisfy the melting point, boiling point and water-immiscibility criteria mentioned above.

The ester oils can suitably be aliphatic or aromatic. Suitable aliphatic ester oils comprise at least one residue containing from 10 to 26 carbon atoms and a second residue of at least 3 carbon atoms up to 26 carbon atoms. The esters may be mono or diesters, and in the latter be derived from a C₃ to C₈ diol or di carboxylic acid. Examples of such oils include isopropyl myristate, isopropyl palmitate and myristyl myristate.

It is especially desirable to employ at least one aromatic ester oil, including especially benzoate esters. Some preferred benzoate esters satisfy the formula Ph-CO—O—R in which R is:

an aliphatic group containing at least 8 carbons, and particularly from 10 to 20 carbons such as from 12 to 15, including a mixture thereof;

or an aromatic group of formula -A-Y-Ph in which A represents a linear or branched alkylene group containing from 1 to 4 carbons and Y represents an optional oxygen atom or carboxyl group.

Particular preferably, the aromatic ester comprises C₁₂₋₁₅ alkyl benzoate.

One further class of ester oils that can constitute a fraction of the ester oils contemplated in the invention compositions comprises natural plant oils, commonly containing glyceride esters and in particular the glyceride triesters of unsaturated C18 aliphatic carboxylic acids, such as linoleic acid, linolenic acid or ricinoleic acid, including isomers such as linolenelaidic acid, trans 7-octadecenoic acid, parinaric acid, pinolenic acid punicic acid, petroselenic acid, columbinic acid and stearidonic acid. Examples of such beneficial natural oils include caster oil, coriander seed oil, impatiens balsimina seed oil, parinarium laurinarium kernel fat, sabastiana brasilinensis seed oil borage seed oil, evening primrose oil, aquilegia vulgaris oil, for and sunflower oil and safflower oil. Such oils can desirably comprise from 1 to 10% by weight of the oil blend.

The ether oil or oils preferably comprise at least one short chain alkyl ether of a polypropylene glycol (PPG), the alkyl group comprising from C2 to C6, and especially C4 and the PPG moiety comprising from 10 to 20 and particularly 14 to 18 propylene glycol units. An especially preferred ether oil bears the INCI name PPG14-butyl ether.

The hydrophobic carrier employed in compositions herein can alternatively or additionally comprise a non-volatile silicone oil, which include polyalkyl siloxanes, polyalkylaryl siloxanes and polyethersiloxane copolymers. These can suitably be selected from non-volatile dimethicones and dimethicone copolyols.

Commercially available non-volatile silicone oils include products available under the trademarks Dow Corning 556 and Dow Corning 200 series (such 350 upwards). Other non volatile silicone oils include that bearing the trademark DC704.

Suitable non-volatile hydrocarbon oils include polyisobutene and hydrogenated polydecene.

Suitable aliphatic alcohols include branched chain alcohols of at least 10 carbon atoms and in many instances up to 30 carbon atoms, particularly 15 to 25, such as isostearyl alcohol, hexyl-decanol octyl-dodecanol and decyl-tetradecanol. Other suitable water-immiscible alcohols include intermediate chain length linear alcohols, commonly containing from 9 to 13 carbon atoms, such as decanol or dodecanol. A further suitable alcohol is benzyl alcohol.

A mixture of two or more of the non-volatile oils can be employed.

The carrier liquid commonly constitutes from 10 to 90% by weight of the base composition, and in many suitably compositions is at least 25% by weight and particularly at least 35% by weight. In many desirable base compositions, the weight proportion of carrier liquid therein is up to 80%, and particularly up to 70%.

The ratio of volatile to non-volatile oils is at the discretion of the formulator to achieve his sensory objectives. If desired, the carrier liquid can be selected within the entire range of from 100% volatile to 100% non-volatile oil, based on the combined weight of volatile and non-volatile oils. However, the weight ratio of volatile to non-volatile oils is more usually selected within the range of from 20:1 to 1:4. It is desirable for the non-volatile oils to provide at least 10% by weight of the carrier liquid and especially at least 20%. In a number of preferred embodiments the weight ratio of volatile to non-volatile oils is selected in the range of from 2:1 to 1:2.

The invention compositions can, if desired, include one or more thickeners or gellants (sometimes called structuring or solidifying agents) to increase the viscosity of or solidify the oil blend in which the particulate materials are suspended as is appropriate for application from respectively roll-on dispensers, soft solid (anhydrous cream) dispensers or stick dispensers. Such thickeners or gellants are selected by the skilled man and enough of them is incorporated to attain the desired viscosity or hardness of the resulting roll-on, lotion or soft solid composition, the actual amount employed taking into account the inherent thickening or gelling capability of the chosen material or combination of materials and their ability to form such a chosen form.

In alternative embodiments, for application from a pressurized aerosol dispenser, the base composition, desirably incorporating a suspension aid, is blended with a propellant.

For application from a roll-on, sufficient thickener is introduced to increase the viscosity of the resultant composition to within the range, typically, of from 1000 to 7000 mPa·s and particularly within 2500 to 5500 mPa·s. Viscosities herein are measured in a Brookfield RVT viscometer equipped with a stirrer TA and Hellipath, rotating at 20 rpm at 25° C.

Herein, the thickener for a roll-on formulation can be selected from suspending agents that can be employed for suspending particulates in a base composition comprising the water-immiscible oil blend, such as particulate silica, especially fumed silica and particulate montmorillonite or bentonite clay, optionally surface treated with a hydrophobic organic compound. Suitable examples are available under the trade names respectively Cab-O-sil and Bentone. Yet other thickeners can comprise oil soluble petrolatum or waxes, such as the waxes described hereinbelow in respect of soft solid or/and sticks. Waxes herein typically are considered to melt at above 40° C. and particularly between 55 and 95° C. Such waxes can include ester waxes, including C12 to C24 linear fatty alcohols, waxes obtained from animals or plants, often after hydrogenation, silicone elastomers and silicone waxes. The thickener system can comprise a mixture of particulate thickeners, a mixture of waxes or a mixture of materials from both. The proportion of thickener or mixture of thickeners is often selected in the range of from 1:30 to 1:12.5 parts per part by weight of oil blend. The viscosity can also be increased by selecting as part of the carrier oil blend, for example from 10 to 20% w/w, relatively viscous non-volatile dimethicone oils or/and hydrogenated polydecene

For use as a soft solid, sufficient thickener is introduced to increase the viscosity of the resultant composition to a hardness of from 0.003 to 0.5 Newton/mm², and commonly from 0.003 or 0.01 up to 0.1 Newton/mm². Hardness can be measured using a Stable Micro systems TA.XT2i Texture Analyser. A metal sphere, of diameter 9.5 mm, is attached to the underside of its 5 kg load cell, and positioned just above the sample surface. Under control of Expert Exceed™ software, the sphere is indented into the sample at an indentation speed of 0.05 mm/s for a distance of 7 mm and reversed to withdraw the sphere from the sample at the same speed. Data comprising time(s), distance (mm) and force (N) is acquired at a rate of 25 Hz. The hardness H at a penetration of 4.76 mm is calculated using the formula

H=F/A

in which H expressed in N.mm⁻², F is the load at the same travelled distance in N and A is the projected area of the indentation in mm⁻².

Stick compositions herein desirably have a hardness as measured in a conventional PNT/Seta penetration test of less than 30 mm, preferably less than 20 mm and particularly desirably less than 15 mm. Many have a penetration of from 7 to 13 or 7.5 to 12.5 mm. The conventional penetration test employed herein, utilises a lab plant PNT penetrometer equipped with a Seta wax needle (weight 2.5 grams) which has a cone angle at the point of the needle specified to be 9°10′+/−15′. A sample of the composition with a flat upper surface is used. The needle is lowered onto the surface of the composition and then a penetration hardness measurement is conducted by allowing the needle with its holder to drop under the combined weight of needle and holder of 50 grams for a period of five seconds after which the depth of penetration is noted. Desirably, the test is carried out at six points on each sample and the results are averaged.

The gellants for forming stick compositions herein are usually selected from one or more of two classes, viz:

non-polymeric fibre-forming gellants sometimes referred to as small molecule gelling agents (viz SMGAs), and waxes, optionally supplemented if desired by incorporation of a particulate silica and/or an oil-soluble polymeric thickener.

The term “wax” is conventionally applied to a variety of materials and mixtures which have similar physical properties, namely that:
they are solid at 30° C. and preferably also at 40° C.;
they melt to a mobile liquid at a temperature above 40° C. and generally below 95° C. and preferably in a temperature range of 55° C. to 90° C.;
they are water-insoluble and remain water-immiscible when heated above their melting point.

Waxes employed herein as gellants, or in other embodiments as thickeners, are often selected from hydrocarbons, linear fatty alcohols, silicone polymers, esters of fatty acids or mixtures containing such compounds along with a minority (less than 50% w/w and often less than 20% w/w) of other compounds. Naturally occurring waxes are often mixtures of compounds which include a substantial proportion of fatty esters.

Waxes usually form crystals in the water-immiscible liquid when it cools from the heated state during processing, often taking the form of needles or platelets depending on the specific wax.

Examples of hydrocarbon waxes include paraffin wax, ozakerite, microcrystalline wax and polyethylene wax, the last named desirably having an average molecular weight of from 300 to 600 and advantageously from 350 to 525.

Linear fatty alcohols commonly contain from 14 to 40 carbon atoms and often from 16 to 24. In practice, most contain an even number of carbon atoms and many comprise a mixture of compounds, even those that are nominally a single one such as stearyl alcohol. Other alcohols include behenyl alcohol

Examples of ester waxes include esters of C₁₆-C₂₂ fatty acids with glycerol or ethylene glycol, which can be isolated from natural products or more conveniently synthesised from the respective aliphatic alcohol and carboxylic acid.

Examples of natural waxes include beeswax, woolwax and spermeceti wax of animal origin, and caster wax, jojoba wax, carnauba wax and candelilla wax which are of vegetable origin. The vegetable waxes are commonly obtained by hydrogenation of the corresponding plant oil, containing triglyceride esters of unsaturated fatty acids. Mineral waxes can be extracted from fossil remains other than petroleum. Montan wax, which is an example of mineral wax, includes non-glyceride esters of carboxylic acids, hydrocarbons and other constituents.

Further waxes employable herein comprise silicone polymer waxes, including waxes which satisfy the empirical formula:

R—(SiMe₂-O—)ₓ—SiMe₂R

in which x is at least 10, preferably 10 to 50 and R represents an alkyl group containing at least 20 carbons, preferably 25 to 40 carbons, and particularly having an average linear chain length of at least 30 carbons.

Other silicone waxes comprise copolymers of dimethicone and alkyloxymethicone, satisfying the general formula:

Y—(SiMe₂-O—)_{y}(Si[OR′]Me-O—)_{z}—Y′

in which Y represents SiMe₂-O, Y′ SiMe₂, R′ an alkyl of at least 15 carbons preferably 18 to 22 such as stearyl, y and z are both integers, totalling preferably from 10 to 50.

Some preferred combinations of waxes include stearyl alcohol with an ester wax such as cater wax, often in a weight ratio of from 10:1 to 3:1.

Waxes useful in the present invention will generally be those found to thicken water-immiscible oils such as cyclomethicones when dissolved therein (by heating and cooling) at a concentration of 5 to 15% by weight.

The second class of thickeners or gellants for sticks for soft solids comprises fibre-forming SMGAs. Such gellants are dissolved in a water-immiscible blend of oils at elevated temperature and on cooling precipitate out to form a network of very thin strands that are typically no more than a few molecules wide. One particularly effective category of such thickeners comprises N-acyl aminoacid amides and in particular linear and branched N-acyl glutamic acid dialkylamides, such as in particular N-lauroyl glutamic acid di n-butylamide and N-ethylhexanoyl glutamic acid di n-butylamide and especially mixtures thereof. Such amido gellants can be employed in anhydrous compositions according to the present invention, if desired, with 12-hydroxystearic acid.

Other amido SMGAs include 12-hydroxystearic acid amides, and amide derivatives of di and tribasic carboxylic acids as set forth in WO 98/27954, including notably alkyl N,N′dialkyl succinamides

Further suitable amido-containing SMGAs are described in U.S. Pat. No. 6,410,003 and satisfy the general formula:

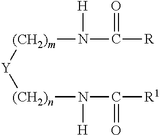

in which m and n are each independently 1 or 0, and Y represents a cyclohexane group substituted in the ortho or para positions by the respectively amido substituents in which R and R′ each represents an aliphatic group containing from 5 to 25 carbons.

A class of amido-containing SMGAs including some specially efficient gellants comprises cyclodipeptide derivatives disclosed in U.S. Pat. No. 7,332,153 having the general formula

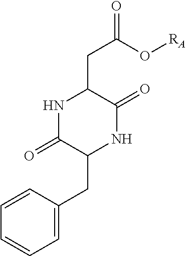

in which R_{A} represents a carbocyclic or heterocyclic group containing not more than 2 rings, other than unsubstituted cyclohexyl. Preferred compounds include such cyclodipeptide derivatives in which the residue R_{A} is derivable from thymol, isopinocamphenol, a 3,5-dialkyl cyclohexanol, carveol.or carvacrol.

A further class of suitable SMGAs is described in U.S. Pat. No. 6,410,001 and satisfies the general formula

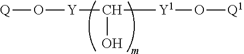

in which Y and Y¹ are each independently —CH₂₋ or >CO, Q and Q¹ are each independently a hydrocarbyl group of at least 6 carbon atoms, and m is from 2 to 24, and particularly gellants in which the substituents terminate in a cyclohexyl ring, optionally substituted by Cl, Br, F, OH, NO₂, O—CH₃, or CH_{3.}

A combination SMGA which is also suitable which is described in U.S. Pat. No. 6,321,841 is a combination of a sterol and a sterol ester and in particular β-sitosterol together with γ-oryzanol preferably in the range of from 3:1 to 1:2.

A further class of SMGAs as described in U.S. Pat. No. 6,248,312 comprises esters of cellobiose and a fatty acid, preferably of 6 to 13 carbon atoms especially 8 to 10 carbon atoms and particularly nonanoic acid. Especially desirably, the cellobiose is esterified, on average, by between 7 and 8 groups, and most desirably adopts the α-anomeric form.

Naturally, a combination of two or more gellants can be employed, such as a wax or mixture of waxes alone, or a mixture of SMGAs alone or a mixture of a wax or waxes plus an SMGA or SMGAs, such as are described hereinabove.

The gellant is often employed in the stick or soft solid composition at a concentration of from 1.5 to 30%, depending on the nature of the gellant or gellants, the constitution of the oil blend and the extent of hardness desired. When an SMGA is employed as the principal gellant, its concentration is typically in the range of from 1.5 to 7.5% w/w for amido gellants or mixtures of them and for 5 to 15% for ester or sterol gellants. When a wax is employed as the principal gellant, its concentration is usually selected in the range of from 10 to 30% w/w, and particularly from 12 to 24% w/w. In many compositions, this corresponds to a weight ratio of the oil ba to the carrier oils selected in the range of 1:30 to 1:2.

If a wax is used which forms a network of fibres, the amount of it may be from 0.5 to 7% by weight of the composition. If a wax is used which does not form such a network, for instance a wax which crystallizes as spherulitic needles or as small platelets, the amount may well be from 2% or 3% up to 10%, 12% or 15% of the composition. Silicone waxes are an example of waxes which crystallize as small platelets.

Some highly desirable compositions comprise in combination a first gellant with a second gellant. The total amount of second gellant may range from 0.5% or 1% of the composition up to 9%, 10% or 15%.

In general, soft solid compositions herein can include one or more of the gellants employed to make a firm stick as described above, but employing a lower concentration of the respective gellant. Thus, the concentration of such gellants is often selected in the range of from 0.5 to 15% w/w of the composition and in many instances from 1 to 10% w/w.

However, it can be especially desirable to employ an oil-soluble polymer as thickening agent for forming a soft solid, for example selected in the range of from 2 to 20% w/w of the composition. Likewise such polymers can be included in stick compositions.

One category of oil-soluble polymer which has been found suitable is a polysaccharide esterified with monocarboxylic acid containing at least 12 carbon atoms, and preferably a dextrin fatty acid ester such as dextrin palmitate or dextrin stearate. Commercial products are available under the trade mark Rheopearl.

A second category of polymer thickener comprises polyamides for example those discussed in U.S. Pat. No. 5,500,209. Such polyamides may be derived from organic diamines containing 2 to 12, preferably 2 to 8 carbon atoms, condensed with di- or poly carboxylic acids containing 4 to 20 carbon atoms per carboxylic acid group, for example VERSAMID 950 derived from hexamethylene diamine and adipic acid. Further polyamides that are copolymers with polysiloxanes are described in U.S. Pat. No. 6,353,076, and particularly its copolymers of Formulae I, II, III, or IV, or as prepared in any of Examples 1 to 5 therein.

A third category of thickening comprises block copolymers of styrene with ethylene propylene and/or butylene available under the trade name KRATON, and particularly styrene ethylene/butylene styrene linear block copolymers. A related category of thickening polymer comprises polymers of alpha methylstyrene and styrene, such as those under the trade name KRISTALEX, eg KRISTALEX F85 having a mean molecular weight of approximately 1200. Yet another thickening polymer comprises alkyl substituted galactomannan available under the trade name N-HANCE AG.

A still further class of thickening polymers co-polymers of vinyl pyrrolidone with polyethylene containing at least 25 methylene units, such as triacontanyl polyvinylpyrrolidone, available under the trade name Antaron WP-660.

Such thickening polymer is often employed in a weight ratio to the oil blend that is selected in the range of from 1:30 to 1:5, taking into account the hardness of the soft solid that is desired, the inherent ability of the chosen polymer to increase viscosity and the presence or otherwise of an additional thickener.

A further class of material which is well suited to forming or contributing to the formation of soft solid compositions comprises silicone elastomers. Such materials are conventionally formed by the hydrosilation of vinyl silicone fluids by hydrosiloxane or MQ hydride fluids. Commonly, for anhydrous compositions, the elastomer is non-emulsifying and especially is a dimethicone/vinyldimethicone cross polymer. Such materials are capable of absorbing a substantial proportion of hydrophobic oils, including cyclomethicones, and are commonly supplied as a dispersion of the active material in either cyclomethicone fluid or a non-volatile oil, typically at a concentration in the region of 10 to 20% by weight. Such elastomers are desirably present at a concentration of from 1 to 10% by weight of the composition.

A thickener especially well suited to forming or contributing to the formation of a soft solid composition comprises a particulate silica and especially a fumed silica. It is desirable to include at least 2% and especially at least 2.5% by weight of the silica in the composition, such as in the range of up to 10% by weight.

The anhydrous compositions can contain one or more optional ingredients, such as one or more of those selected from those identified below.

Optional ingredients include wash-off agents, often present in an amount of up to 10% w/w to assist in the removal of the formulation from skin or clothing. Such wash-off agents are typically nonionic surfactants such as esters or ethers containing a C₈ to C₂₂ alkyl moiety and a hydrophilic moiety which can comprise a polyoxyalkylene group (POE or POP) and/or a polyol.

The compositions herein can incorporate one or more cosmetic adjuncts conventionally contemplatable for cosmetic solids or soft solids. Such cosmetic adjuncts can include skin feel improvers, such as talc or finely divided (i.e. high molecular weight) polyethylene, i.e. not a wax, for example Accumist™, in an amount of 1 up to about 10%; a moisturiser, such as glycerol or polyethylene glycol (mol wt 200 to 600), for example in an amount of up to about 5%; skin benefit agents such as allantoin or lipids, for example in an amount of up to 5%; skin cooling agents, such a menthol and menthol derivatives, often in an amount of up to 2%, all of these percentages being by weight of the composition. A further optional ingredient comprises a preservative, such as ethyl or methyl paraben or BHT (butyl hydroxy toluene) such as in an amount of from 0.01 to 0.1% w/w.

Aerosol base compositions desirably additionally comprise a suspending aid, sometimes called a bulking agent which is typically a powdered silica or a layered clay, such as a hectorite, bentonite or montmorillonite in powder form. The layered clay is optionally hydrophobically surface treated. Particularly suitable surface treated clays are available under the trade mark Bentone, such as Bentone 38. The suspending aid often constitutes from 0.5 to 4% by weight of the base aerosol composition. Aerosol base compositions desirably also can contain a swelling aid to assist swelling of the layered clay, often selected in a proportion of from 0.005 to 0.5% by weight of the aerosol base composition and particularly in a weight ratio to the clay of from 1:10 to 1:75. Suitable swelling aids include especially propylene carbonate and triethyl citrate.

The invention compositions herein can additionally contain a water-soluble polymer comprising a Bronsted acid group that cooperates synergistically with the aluminium or aluminium/zirconium antiperspirant active to enhance antiperspirant efficacy. Such a material is referred to in U.S. Pat. No. 6,616,921 as a co-gellant (because it assists the antiperspirant active to gel in eccrine pores) and is described therein. Preferred examples of such a co-gellant are polymers having a molecular weight of at least 50,000 derived at least in part from maleic acid or maleic anhydride, such as Gantraz™ AN119, AN139 or AN169. The co-gellant is often selected in a weight ratio to the aluminium or aluminium/zirconium salt of from 1:15 to 1:2.

The invention compositions herein can additionally comprise an encapsulated or non-encapsulated fragrance, or a mixture of both encapsulated and non-encapsulated fragrance, which can be the same or different. The total weight of fragrance in the base composition is often selected in the range of from 0.01 to 4% and particularly from 0.1 to 1.5%. The weight ratio of encapsulated to non-encapsulated fragrance, when both are resent, is often in the range of from 5:1 to 1:5.

Herein, the ingredient that is susceptible to photo-destruction by visible/UV light is one or a mixture selected from:
Molecules which produce singlet oxygen under exposure to light in the range 290-740 nm, preferably up to 700 nm and oxygen with a quantum yield greater than 0.05 in an organic solvent;
Molecules comprising aromatic heterocycles
Phenols which lack a tertiary butyl group that would serve to stabilise the phenol,
Biological molecules: Vitamins, Co-enzymes, Enzymes and proteins and Unsaturated aliphatic olefinically unsaturated fatty carboxylic acids.

Preferably, in some desirable embodiments, such an ingredient is absent or has a principal coefficient of extinction that is more than 150 nm from the colour the photostable pigment employed herein.

In a further aspect of the present invention there is provided a non-therapeutic antiperspirant composition comprising
an antiperspirant active at a concentration of at least 5% by weight,
a carrier liquid for the antiperspirant active and
a polycyclic organic pigment selected from Cu phthalocyacnine, metal free phthalocyanine, quinacridone, perylene, diketopyrrolo-pyrrole, thioindigo, anthrapyrimidine, flavanthrone, pyranthrone, dioxazine and quinophthalone pigments dispersed through the carrier liquid,

which composition further comprises at least 0.1% by weight of a fragrance that is entrapped and/or itself comprises fragrance components in a weight ratio of from 1:9 to 3:1 of components having a boiling point at 1 bar pressure of up to 250° C. to components having a boiling point at 1 bar pressure of greater than 250° C.

Herein, by the term entrapped in the context of a fragrance is meant that the fragrance is held releasably in an entrapment material, by a physical or chemical attraction. The entrapment material may comprise capsules or microcapsules having a shell surrounding a core of the fragrance, or/and a matrix comprising voids which can be filled by the fragrance. The capsules can be pressure or moisture-sensitive, which is to say the fragrance can be released by contact with water or by the capsules being subjected to impact or sustained pressure. Suitable encapsulation materials are described in EP1289485.on page 8 paragraphs 0052 and 0053, including dextrins, gum acacia and waxes. Other suitable encapsulating materials comprise aminoplasts (eg melamine-formaldehyde), as described for example in U.S. Pat. No. 3,516,941 or U.S. Pat. No. 6,261,483, or gelatin as described for example in U.S. Pat. No. 6,045,835. The entrapment material can comprise an absorbent, such as an aluminosilicate (eg a zeolites), a clay, eg bentonite, kaolinite, hectorite or Laponite™ synthetic hectorite or activated alumina.

A further class of entrapment material which can be especially desirable comprises a cyclic oligosaccharide and in particular α or preferably β cyclodextrins. Pro-fragrances are examples of entrapment materials offering chemical attraction, as described for example in WO 98/47477 or WO98/07405.

The entrapment material is typically at least 10% by weight of the combined weight of fragrance and entrapment material and often up to 90% by weight, the actual proportion being dependent on the type of entrapment material and the preference of the formulator.

The boiling point of individual fragrance components can be identified from reference works such as in Perfume and Flavor Chemicals, Steffen Arctander (1969), the International Cosmetic Ingredient Dictionary and Handbook. Other or more recent fragrance chemicals are disclosed by searching for their name in websites such as “thegoodscentscompany.com”

Fragrance components having a boiling point of up to 250° C. (lower BP components) include anethol, methyl heptine, carbonate, ethylacetoacetate, paracymene, nerol, decyl aldehyde, 2,6-nonadienal, p-cresol, methylphenylcarbinyl acetate, α-ionone, β-ionone, undecylenic aldehyde, undecyl aldehyde nonyl aldehyde octyl aldehyde, benzyl acetate, camphor, carvone, borneol, bornyl acetate, eucalyptol, linalool, iso-amyl acetate, thymol, carvacrol, limonene, iso-amyl alcohol, α-pinene, β-pinene, α-terpineol, citronellol, dimethylbenzylcarbinol, citral, citronellal nitrile, dihdromyrcenol, geraniol, geranyl acetate, hydroxycitronellal, linalyl acetate, tetrahydrolinalool and verdox. Examples of fragrance components having a boiling point of over 250° C. (higher BP components) include ethylmethylglycidate, ethyl vanillin, heliotropin, indol, methyl anthranilate, vanillin, amyl salycilate, coumarin, ambrox, bacdanol, benzyl salycilate butyl anthranilate, cetalox, ebanol, lillial, γ-undecalactone, γ-decalactone, iso-eugenol, lyral, florhydral, eugenol, amyl cinnamic aldehyde, hexyl salicylate, sandalone, galaxolide and pentalide. Mixture of 2 or more components within and between the two classes of components can be employed. Indeed it is desirable to employ at least 20 fragrance components including at least 5 from each of the two classes.

In many suitable fragrance blends for employment herein, the weight ratio of lower BP components to higher BP components is often selected in the range of from 1:4 to 3:2.

The fragrance herein can employ a mixture of entrapped and non-entrapped fragrance, such as in a weight ratio of the former to the latter of from 4:1 to 1:4.

The compositions in this second aspect can employ the variations in composition described hereinbefore in respect of the first aspect.

Herein unless the context demands otherwise, all weights, % s, and other numbers can be qualified by the term “about”.

The invention compositions can be made by the methods hitherto suggested for the preparation of antiperspirant compositions not containing the invention photostable pigment.

One convenient process sequence for preparing a stick or soft composition according to the present invention comprises first forming a solution of the structurant combination in the water-immiscible liquid or one of the water-immiscible liquids. This is normally carried out by agitating the mixture at a temperature sufficiently high that all the structurants dissolve (the dissolution temperature) such as a temperature in a range from 70 to 140° C. Any oil-soluble cosmetic adjunct can be introduced into oil phase, either before or after the introduction of the gellants. However, the fragrance oil, be it encapsulated or free, is commonly the last ingredient to be incorporated into the composition, after the antiperspirant active on account of its sensitivity often to elevated temperature. Commonly the resultant structurant solution is allowed to cool to a temperature that is intermediate between that at which the gellants dissolved and the temperature at which it would set, often reaching a temperature in the region of 60 to 90° C.

In some routes, the carrier oils can be mixed together prior to introduction of the gellants and the antiperspirant or deodorant active. In other preparative routes, it is desirable to dissolve all or a fraction of the gellants and especially for amido gellants in a first fraction of the composition, such as a branched aliphatic alcohol, e.g. isostearyl alcohol or octyldodecanol, optionally in conjunction with an alcohol having some water-miscibility and boiling point above the dissolution temperature of the amido gellant in the alcoholic fluid. This enables the remainder of the carrier fluids to avoid being heated to the temperature at which the structurants dissolve or melt. Such a process commonly involves mixing the fractions intensively in for example a “Sonolator”™. In the invention compositions, the fragrance capsules are most desirably introduced after any intensive mixing step. The proportion of the carrier fluids for dissolving the structurants is often from 25 to 50% by weight of the carrier fluids.

In said other preparative routes the particulate material is introduced into preferably a second fraction of the carrier oils, for example silicone and/or ester and/or hydrocarbon oils and thereafter, and thereafter the first fraction containing dissolved structurant and second fraction containing suspended particulate material are mixed at a temperature above that at which the composition gels, and often from 5° C. to 30° C. above the regular setting temperature of the composition, dispensing containers are filled and cooled or allowed to cool to ambient temperature. Cooling may be brought about by nothing more than allowing the container and contents to cool. Cooling may be assisted by blowing ambient or even refrigerated air over the containers and their contents.

Suspension roll-on compositions herein can be made by first charging a mixing vessel equipped with agitation means such as a stirrer or a recycle loop with the oils simultaneously or sequentially, and thereafter charging the vessel with the antiperspirant/deodorant active ingredient, the thickener and any optional ingredient and heating the composition to the extent necessary to dissolve any organic thickener in the oil blend. Thereafter, the resultant fluid composition is discharged into roll-on dispensers through the open top and the ball (or more unusually cylindrical roller) inserted and the cap fitted.

Aerosol products herein comprise a base composition comprising an antiperspirant and/or deodorant active suspended in an oil blend together with the fragrance capsules, suspending agent and optional ingredients that is blended with a propellant, commonly in a weight ratio of blend to propellant of from 1:1 to 1:15, and in many formulations from 1:3 to 1:9. The propellant is commonly either a compressed gas or a material that boils at below ambient temperature, preferably at below 0° C., and especially at below −10° C. Examples of compressed gasses include nitrogen and carbon dioxide. Examples of low boiling point materials include dimethylether, C₃ to C₆ alkanes, including in particular propane, butanes and isobutane, optionally further containing a fraction of pentane or isopentane, or especially for use in the USA the propellant is selected from hydrofluorocarbons containing from 2 to 4 carbons, at least one hydrogen and 3 to 7 fluoro atoms.

Aerosol products can be made in a conventional manner by first preparing a base composition, charging the composition into the aerosol can, optionally introducing the fragrance into the can after the base composition, (late fill addition), fitting a valve assembly into the mouth of the can, thereby sealing the latter, and thereafter charging the propellant into the can to a desired pressure, and finally fitting an actuator on or over the valve assembly together with an overcap if the can does not employ through the cap spraying.

Product Dispenser

Although the stick composition could be formed into an extruded bar and wrapped and sold in that form, generally it is desirable to house the composition in a stick dispenser that conventionally comprises a barrel one at one end, and a platform located beneath the open end adapted to propel the stick composition out of the barrel through the open end. The means for propulsion can comprise an opening at a second end of the barrel remote from the first end through which a finger could be inserted to come into contact with the underside of the platform, or more usually is rotor wheel at the base of a barrel on which is mounted a threaded spindle that extends through a correspondingly threaded aperture in the platform. The barrel engages the platform so as to prevent rotation of the latter, so that when the rotor wheel and spindle are rotated, the platform is advanced or retracted to or from the open end. Examples of suitable dispensers are described, for example, in U.S. Pat. No. 4,232,977, U.S. Pat. No. 4,605,330, WO09818695, WO09603899, WO09405180, WO09325113, WO09305678, EP1040445, U.S. Pat. No. 5,997,202, U.S. Pat. No. 5,897,263, U.S. Pat. No. 5,496,122, U.S. Pat. No. 5,275,496, U.S. Pat. No. 6,598,767, U.S. Pat. No. 6,299,369, or WO 2002/03830. The dispensers or moulds into which the stick composition is introduced can be made from thermoplastic material, such as polyethylene and commonly contain from 10 to 100 g composition, such as in the range of from 15 to 25 g for small or sample sticks and from 40 to 80 g for regular or large sticks.

Dispensers for soft solids are generally similar to those for firm sticks, except that mostly, the open end of the barrel is fitted with a top wall, often domed, defined at least one and typically a series of narrow apertures through which the soft solid can be extruded by gentle pressure. The same type of advance mechanism can be employed as for firm sticks. Suitable dispensers for soft or semi-solids are exemplified in U.S. Pat. No. 4,865,231, U.S. Pat. No. 5,000,356, U.S. Pat. No. 6,116,803, U.S. Pat. No. 5,961,007, WO9851185, EP0312165, WO0019860, EP0709041, EP858271, U.S. Pat. No. 5,573,341, U.S. Pat. No. 5,725,133, U.S. Pat. No. 5,248,213, U.S. Pat. No. 6,398,439 or U.S. Pat. No. 6,450,716.

Commonly, the dispenser for sticks or soft solids is moulded from a thermoplastic such as polypropylene or polyethylene.

A suitable applicator for dispensing a roll-on composition comprises a bottle having a mouth at one end defining a retaining housing for a rotatable member, commonly a spherical ball or less commonly a cylinder which protrudes above the top wall of the bottle. Suitable applicators are described for example in EP1175165, or WO2006/007987. The bottle mouth is typically covered by a cap, typically having a screw thread that cooperates with a thread on the housing or in an innovative design by a plurality of staggered bayonet/lug combinations. Although in past times the bottle commonly was made from glass with a thermoplastic housing mounted in the mouth of the bottle, most roll-on dispensers are now made entirely from thermoplastic polymers.

A suitable dispenser for an aerosol composition comprises a can, usually made from steel or aluminium, often having a coated interior to prevent contact between the can contents and the can wall, which contents can be vented to the exterior through a dip tube leading via a valve that is openable and closable by an actuator, into a spray channel terminating in a spray nozzle. Suitable dispensers are described, for example in EP1219547, EP1255682, or EP1749759.

It is advantageous to employ the invention compositions in a dispenser that has a transparent or translucent wall or window. That enables the user to view the formulation within the dispenser. This is particularly appropriate for non-pressurised dispensers, such as dispensers for dispensing sticks, soft solids or roll-ons. It is particularly suitable for a stick composition, in that the dispenser may comprise a translucent cap, enabling the top surface of the composition to be exposed to light.

Having summarised the invention and described it in more detail, together with preferences, specific embodiments will now be described more fully by way of example only.

### EXAMPLES

In the Examples herein, the pigments are denoted by their colour index name.

### Example 1

Suspension antiperspirant sticks were created with the following formulation:

TABLE 1

| Material | % weight |
|---|---|
| Aluminium Zirconium | 20 |
| Tetrachlorohydrex GLY | |
| Cyclomethicone (D5) | 26.3 |
| PPG-14 Butyl Ether | 9.5 |
| C12-15 Alkyl Benzoate | 15 |
| Dimethicone | 1 |
| Polyethylene mw 450 | 1 |
| Stearyl alcohol | 18 |
| Hydrogenated Castor oil MP80 | 3.5 |
| Helianthus Annuus Seed Oil | 0.5 |
| Butyl Hydroxy Toluene | 0.05 |
| Steareth-100 | 0.5 |
| Silica | 0.75 |
| PEG-8 | 2 |
| Parfum | 1.2 |
| 1% Pigment premix | 0.7 |

The invention and comparison compositions containing the specified pigments in Example 2 were made by the following method:

For each pigment, a premix was formed by suspending the selected pigment in cyclomethicone at a concentration of 1% by weight.

The oils were blended, heated together with the gellants (stearyl alcohol, hydrogenated castor oil and polyethylene wax) to a temperature of about 85° C. and maintained with stirring at that temperature until the waxes had dissolved. The resultant composition was allowed to cool to about 75° C. and the particulate solids (AZAG and silica) and the premix of pigment were introduced with vigorous stirring. Finally the perfume was added with stirring and the resultant composition that was still mobile was poured into a conventional plastic stick dispenser of barrel volume approximately 45 g through a base aperture and when the contents had solidified, the dispenser was inverted.

The Parfum employed in this Example had a ratio of lower boiling point to higher boiling point components that fell within the weight ratio range of from 1:9 to 3:1.

### Example 2

Following preparation and cooling, the sticks of example 1 were irradiated in simulated outdoor Florida sunlight for 45 hours at 20% RH, 0.35 W/m² (340 nm) (IR filter) in a weatherometer. The sticks were placed with top of the barrel facing the light, and a clear plastic top placed on the sticks. Subsequent to irradiation the stick colour was measured using a Reflectometer (UV excluded) and the compared to un-irradiated sticks. The % dye remaining was calculated from the reflectance spectra via the Kulbeka Munk equation:

K/S=(1−R)²/(2R)

Where K/S is the remission function and R is the % reflectance/100. K/S is proportional to the amount of pigment in the formulation. By comparison of irradiated and unirradiated values, taking into account the base line (values when no pigment is present) the % dye remaining is obtained.

The results are summarised in Table 2 below

TABLE 2

| C.I. Pigment name | Structure | % dye remaining |
|---|---|---|
| Pigment Blue 15 | 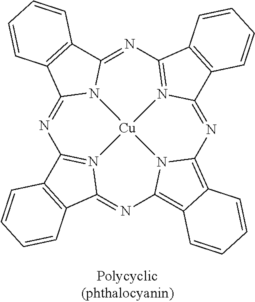 | no dye loss measurable |
| Pigment Violet 19 | 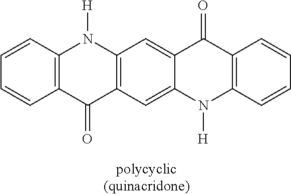 | no dye loss measurable |
| Pigment Violet 23 | 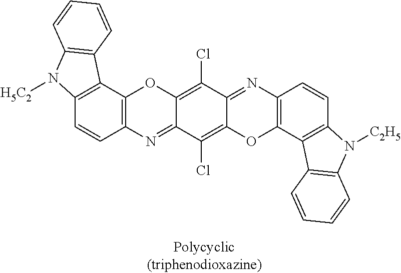 | no dye loss measurable |
| Pigment Red 5 Comparative Example | 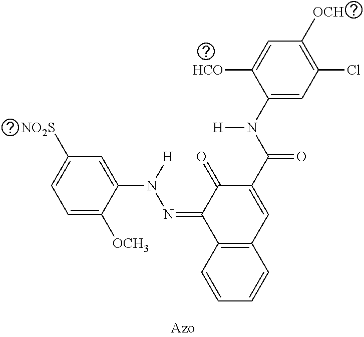 | 15 |
| Pigment Red 49:1 Comparative Example | 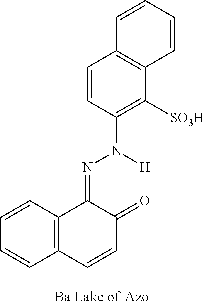 | 6 |
| Pigment Red 68 Comparative Example | 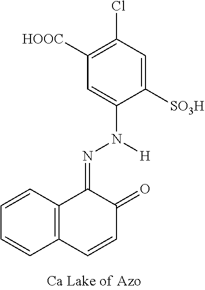 | 14 |
| Pigment Red 51 Comparative Example | 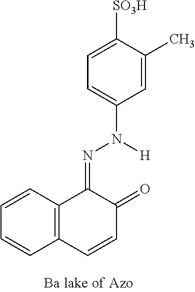 | 5 |
| Pigment Red 273 Comparative Example | 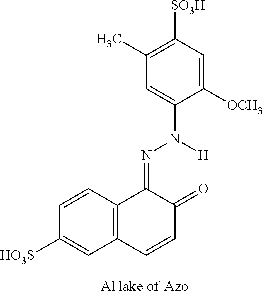 | 15 |

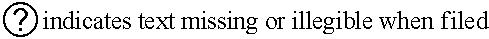

From the table above, it is self-evident that the pigments according to the invention were photostable (and hence resistant to photodegradation) whereas comparative azo pigments were susceptible to photo-degradation

1. A non-therapeutic antiperspirant composition comprising
At least 5% by weight of an antiperspirant active,
a carrier liquid for the antiperspirant active and
a polycyclic organic pigment selected from Cu phthalocycanine, metal free phthalocycanine, quinacridone, perylene, diketopyrrolo-pyrrole, thioindigo, anthrapyrimidine, flavanthrone, pyranthrone, dioxazine and quinophthalone pigments dispersed through the carrier liquid,
which composition is free from an ingredient that is susceptible to photo-degradation by visible and/or UV light having a peak coefficient of extinction within 150 nm of the colour of the pigment or contains less than 1 part by weight of said susceptible ingredient per 2 parts by weight of said pigment.

2. A composition according to claim 1 in which the pigment is a Cu phthalocycanine, metal free phthalocycanine, quinacridone or triphenodioxazine.

3. A composition according to claim 2 in which the pigment is one or more of Pigment Violet 23, Pigment Blue 60, Pigment Blue 64, Pigment Orange 43, Pigment blue 66, Pigment Blue 63, Pigment Violet 36, Pigment Violet 19, Pigment Red 122, Pigment Blue 16, Pigment Blue 15, Pigment Blue 15:1, Pigment Blue 15:2, Pigment Blue 15:3, Pigment Blue 15:4, Pigment Blue 15:6 and Pigment Green 7.

4. A composition according to claim 1 in which the composition contains from 0.001 to 1% of the pigment.

5. A composition according to claim 1 in which the composition is anhydrous.

6. A composition according to claim 1 in which the antiperspirant active is present in a weight ratio to the selected pigment of from 100:1 to 600:1.

7. A composition according to claim 1 in which the composition is a stick having a hardness of less than 15 mm penetration in a conventional PNT/Seta penetration test.

8. A composition according to claim 1 in which the composition is contained within a dispenser having a transparent or translucent wall or window.
